(19) United States
(12) Patent Application Publication
(10) Pub. No.: US 2010/0191177 A1
(43) Pub. Date: Jul. 29, 2010

(54) DEVICE FOR ASPIRATING FLUIDS

(75) Inventors: Tom S. Chang, Los Angeles, CA (US); Friedrich Ho, Mountain View, CA (US); Stanley R. Conston, San Carlos, CA (US); Ronald K. Yamamoto, San Francisco, CA (US)

Correspondence Address: Weaver Austin Villeneuve & Sampson LLP, P.O. BOX 70250, OAKLAND, CA, 94612-0250, US

(73) Assignee: iScience Interventional Corporation, Menlo Park, CA (US)

(21) Appl. No.: 12/359,169

(22) Filed: Jan. 23, 2009

(51) Int. Cl. A61F 9/00

(52) U.S. Cl. 604/22

(57) ABSTRACT

Surgical devices are provided for aspiration of the subretinal fluid (SRF) of the eye in a retinal detachment that allows re-apposition of the sensory retina to the underlying RPE. The device is connected to a vacuum source, introduced into the posterior chamber through a sclerostomy port and placed against the detached retinal tissue. The device pulls on and captures the surface of the sensory retina, causing a micro needle to pierce through the tissue. As the sensory retina is captured and held in place by the vacuum, a protected pocket is created and the tissue is prevented from folding onto itself and occluding the micro needle tip.

### CROSS-REFERENCE TO RELATED APPLICATION

This application is related to co-pending, commonly assigned Ser. No. ______, filed on an even date herewith, entitled “Subretinal Access Device” in the names of Ho, Friedrich; Conston, Stanley R. and Yamamoto, Ronald.

### FIELD OF THE INVENTION

The present invention relates to devices for aspiration of the subretinal fluid (SRF) of the eye in a retinal detachment that allows re-apposition of the sensory retina to the underlying RPE.

### BACKGROUND OF THE INVENTION

A retinal detachment occurs when subretinal fluid (SRF) causes separation between the sensory retina and the supporting outer tissues, which consist of the retinal pigment epithelium (RPE) and choroid. Typically, retinal detachments are caused when a full-thickness defect in the sensory retina allows for SRF to access the subretinal space. This SRF is derived from liquefied vitreous humor (the transparent gel that occupies the posterior segment of the eye), and full-thickness defects may be defined by either a tear or hole in the retina. Additionally, a retinal detachment can be caused when the sensory retina is pulled away from the RPE due to the tractional forces of the vitreous body. In this case, the SRF may be derived from the capillaries in the choroid and can gain access to the subretinal space through the RPE. Retinal detachments may form spontaneously due to an eye or head injury. Existing pathologies may also contribute to retinal detachments, such as diabetic retinopathy.

Retinal detachments usually require immediate surgical repair. If left untreated, liquefied vitreous can continue to enter the subretinal space through a tear or vitreal traction can continue to apply separation forces on the sensory retina. Chronic separation between the sensory retina and the underlying RPE can deprive the sensory retina of nutrients and oxygen, causing the sensory retina to atrophy with resultant vision loss.

Current methods to treat retinal detachments by re-apposition of the sensory retina to the RPE and choroid include scleral buckling, pneumatic retinopexy, and vitrectomy with the use of tamponading agents. These treatments are often accompanied by cryopexy or laser photocoagulation to seal retinal tears. Each of the above procedures does not provide immediate re-apposition of the detached retina to the underlying tissues. Aspiration of the SRF to provide immediate re-apposition of the detached retina may provide greater reattachment efficacy and reduced healing time. Failure to provide immediate re-apposition can result in the use of uncomfortable implants that limit the range of eye motion and produce long reabsorption rates of the SRF by the tamponade effect.

Aspiration of the SRF using a polymer micro needle to cross the sensory retina from the interior and draining the fluid with an external vacuum source is one approach. However, this method can fail due to the properties of the sensory retinal tissue. By nature, the sensory retina and the RPE are very flexible and conformal, and therefore the tissues may be directed by the vacuum source into the opening of the needle, occluding the needle and preventing aspiration of the SRF. Another failure mode may be attributed to kinking of the micro needle. While accessing the subretinal space, the micro needle may be bent when encountering tissues, kinking the shaft and reducing its effective vacuum and ability to aspirate the SRF. There is a need for devices that address the issue of occlusion via aspiration of the tissues into the device lumen.

The present invention provides devices that allow for aspiration of the SRF in a retinal detachment using an ab-interno approach in conjunction with sclerostomy port systems. Use of the devices allows aspiration and removal of the SRF that overcomes the previously described failure modes, thus allowing for immediate re-apposition of the sensory retina to the underlying RPE without any tamponading agents or implants. In addition, the devices of the invention may be used to provide re-apposition of the sensory retina as an adjunctive means to other forms of retinal detachment treatment to improve reattachment and healing.

### SUMMARY

The present invention provides a surgical device for use in the eye comprising:

a first elongated tubular member having a proximal and a distal end and a lumen passing from the proximal end to the distal end, preferably sized appropriately to fit through a conventional sclerostomy port;

a second elongated tubular member having a proximal end and a distal end having a pointed tip, disposed within the lumen of the first tubular member, the second elongated tubular member having a passage therethrough from its proximal end to its distal end;

an annular space within the lumen of the first elongated tubular member, annularly surrounding the second elongated tubular member wherein the passage and the annular space are in communication;

the distal end of the first elongated tubular member being open-ended and adapted to be placed in contact with a tissue surface whereby upon reduction of pressure within the annular space, the distal end of the first elongated tubular member seals to the tissue and the pointed tip penetrates the tissue and aspirates fluid beneath the tissue into the passage from the distal end of the second elongated tubular member.

In one embodiment the passage in the second elongated tubular member may be in communication with a device for aspirating fluids, suspensions, viscous solids or gases, through the passage. The device for aspiration may comprise a syringe or a surgical vacuum source.

In one embodiment the distal end of the second elongated tubular member extends beyond the open distal end of the first elongated tubular member. Typically the second elongated tubular member extends beyond the open distal end of the first elongated tubular member by about 0.005 inch to about 0.125 inch.

In one embodiment the device further comprises one or more fenestrations in the second elongated tubular member that extends beyond the open distal end of the first elongated tubular member. Typically the fenestrations have a maximum diameter of in the range of about 0.0005 inch to about 0.005 inch. Typically the centers of one or more fenestrations are a distance from the distal end of the second elongated tubular member in the range from about 0.001 inch to about 0.01 inch.

In another embodiment the device further comprises a blocking member disposed in the annular space at the distal end of the device, the blocking member having a configuration sufficient to substantially prevent the ingress of tissues into the annular space through the open distal end without preventing fluid flow through the annular space. The blocking member may typically comprise a coil, a loop or a perforated sheet. In a perforated sheet the perforations typically have average diameters in the range from about 0.0001 inch to about 0.005 inch.

In one embodiment the device further comprises a stiffening member disposed within the lumen. The stiffening member typically comprises a wire.

In another embodiment the device further comprises a third hollow tubular member in communication with the annular space.

In one embodiment the device further comprises a tissue guard disposed within the passage of the second elongated tubular member that extends beyond the open distal end of the first elongated tubular member. The tissue guard may typically comprise a wire loop or coil. In one embodiment the wire has an atraumatic tip.

In another embodiment the device further comprised a tissue guard disposed external to the second elongated tubular member that extends beyond the open distal end of the first elongated tubular member. The tissue guard may be collapsible. The tissue guard is typically disposed up to about 0.01 inches from the distal end of the second elongated tubular member that extends beyond the open distal end of the first elongated tubular member. The tissue guard may comprise a balloon or slits that expand when compressed. In some embodiments the tissue guard is expandable by activation.

In one embodiment the device further comprises a sensor for activating the tissue guard. The tissue guard may be activated mechanically or electrically by the sensor. In some embodiments the tissue guard is adapted to activate to automatically expand upon penetration into the subretinal space.

### BRIEF DESCRIPTION OF THE DRAWINGS

FIG. 1 is a schematic diagram of a retinal detachment aspiration cannula device according to the invention.

FIG. 2 is a schematic diagram of the working device tip according to the invention.

FIG. 3 is a schematic diagram of an embodiment of a device according to the invention at the proximal portion of the main shaft.

FIG. 4 is a schematic diagram of a distal tip of a device according to the invention comprising a tissue blocking mechanism flush with distal tip of the main shaft.

FIG. 5 is a schematic diagram of a distal tip of a device according to the invention comprising a tissue blocking mechanism protruding from the distal tip of the main shaft.

FIG. 6 is a schematic diagram of a distal tip of a device according to the invention comprising a micro needle lumen with an increased diameter within the main shaft.

FIG. 7 is a schematic diagram of a distal tip of a device according to the invention comprising features to facilitate entry into the subretinal space.

FIG. 8 is a schematic diagram of a distal tip of a device according to the invention comprising a stiffening member disposed within the lumen of the micro needle.

FIG. 9 is a schematic diagram of a cannula device according to the invention comprising an infusion line.

FIG. 10 is a schematic diagram of a preferred embodiment of a cannula device according to the invention.

FIG. 11 is a schematic diagram of another preferred embodiment of a cannula device according to the invention.

FIG. 12 is a schematic diagram of another preferred embodiment of a cannula device according to the invention.

FIG. 13 is a schematic diagram of a cannula device according to the invention with a guarded tip having a wire loop.

FIG. 14 is a schematic diagram of a cannula device according to the invention with fenestrations near the distal tip.

FIG. 15 is a schematic diagram of a cannula device according to the invention with fenestrations near the distal tip and a guarded tip having a wire loop.

FIG. 16 is a schematic diagram of a cannula device according to the invention with an external tissue guard near the distal tip.

FIG. 17 is a schematic diagram of a cannula device according to the invention with a mechanical deployment mechanism for an external tissue guard

FIG. 18 is a schematic diagram of a cannula device according to the invention deployed through a sclerostomy port and in communication with a retinal detachment.

### DESCRIPTION OF THE PREFERRED EMBODIMENTS

The present invention provides surgical devices for aspirating SRF from the subretinal space in a retinal detachment. The devices comprise features that advantageously avoid potential failure methods associated with previous attempts to aspirate the SRF using a micro needle. The failure methods include occlusion of the micro needle by the sensory retina and the RPE, trauma to the retina and kinking of the micro needle.

The present invention provides a device for aspirating subretinal fluid (SRF) when the subretinal space is accessed from the interior of the globe of the eye through a conventional sclerostomy port system.

It is preferred to introduce the device to the posterior chamber with the use of a sclerostomy port. The sclerostomy port is introduced through the sclera at the pars plana to provide access to the posterior chamber. The port provides surface stabilization, sealing to maintain posterior chamber pressure and the ability to interchange surgical tools. Sclerostomy port systems are commercially available to provide access for devices typically from 20 to 25 gauge in diameter.

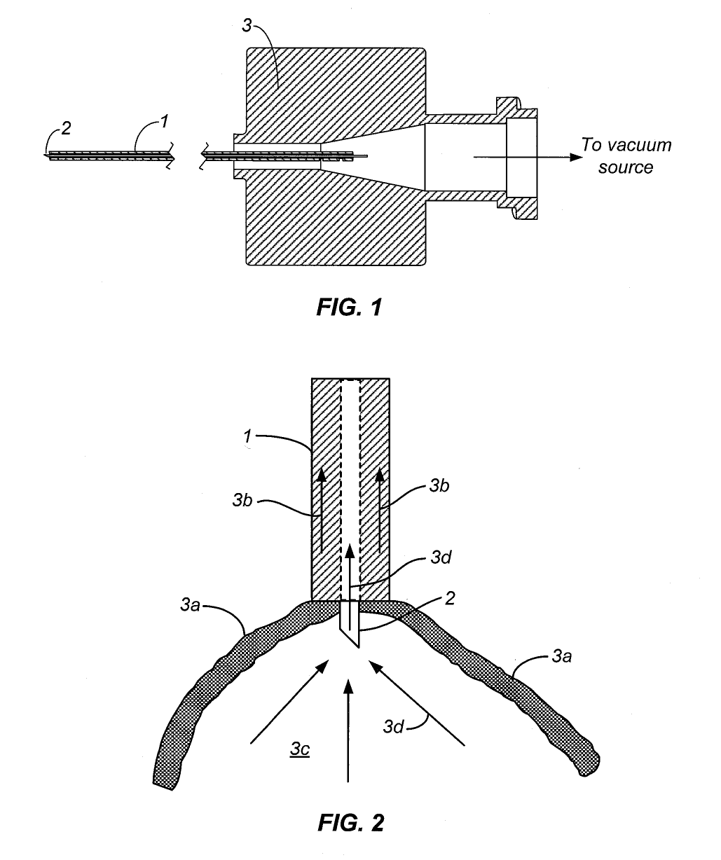

As shown in FIG. 1, one embodiment of the device comprises a tubular member 1, a second smaller tubular member 2, and a connection device for communication of fluid or gas exchange 3. Furthermore, the second smaller tubular member may or may not be hollow, in that aspiration may comprise the use of wicking materials or materials that utilize capillary action to remove fluid. In addition, the invention comprises means to safely stabilize and prevent delicate retinal tissues from blocking the communication flow pathway during use.

In general, a device according to the invention comprises a first elongated tubular member having a proximal and a distal end and a lumen passing from the proximal end to the distal end;

a second elongated tubular member having a proximal end and a distal end having a pointed tip, disposed within the lumen of the first tubular member, the second elongated tubular member having a passage therethrough from its proximal end to its distal end;

an annular space within the lumen of the first elongated tubular member, annularly surrounding the second elongated tubular member wherein the passage and the annular space are in communication;

the distal end of the first elongated tubular member being open-ended and adapted to be placed in contact with a tissue surface whereby upon reduction of pressure within the annular space, the distal end of the first elongated tubular member seals to the tissue and the pointed tip penetrates the tissue and aspirates fluid material beneath the tissue into the passage from the distal end of the second elongated tubular member.

The tissue surface contacted with the distal end of the first elongated tubular member to form a seal will be in the interior of the eye once the device is adapted to access the subretinal space from the interior of the globe of the eye. This is facilitated by entry through a conventional sclerostomy port system.

In a first embodiment, as shown in FIG. 1, a hollow tubular outer member, or main shaft 1, typically has a useful outer diameter in the range of about 0.010″ to about 0.050″, for compatibility with conventional sclerostomy ports. The second smaller hollow tubular member, or micro needle 2, is used for SRF aspiration and is placed concentrically within the main shaft. When vacuum is applied to the annular space created between the main shaft and the micro needle, the vacuum present in the annular space retains the retinal tissues and prevents occlusion of the distal tip of the micro needle.

The distal tip of the micro needle typically extends beyond the distal tip of the main shaft for a distance in the range of about 0.0015″ to about 0.125″ to accommodate the variation in retinal thickness and the depth of retinal detachment. The micro needle is disposed coaxially within and along the length of the main shaft, and has a typical useful outer diameter of about 0.0020″ to about 0.0070″ to minimize injury to the retina when the micro needle pierces the retinal tissue to access the subretinal space. When vacuum is applied to the micro needle, the SRF is aspirated. When the connection device 3 is attached to a vacuum source, a vacuum level determined by the user is applied to both the micro needle and the annular space between the main shaft and micro needle. The vacuum level may be typically varied from 10-760 mm Hg depending upon the amount and viscosity of the fluid being aspirated. The device 3 may be adapted to aspirate fluids, suspensions, viscous solids or gases. The relative vacuum level in the annular space and the micro needle lumen may be proportioned appropriately by the design of the respective flow pathways. Alternatively, two separate vacuum sources may be used for the outer annular space and the micro needle lumen.

Referring to FIG. 2, when the device is connected to a vacuum source and the distal end of the device is placed against the retinal tissue 3a, the outer annular vacuum, represented by arrows 3b, pulls on and captures the surface of the sensory retina, causing the micro needle 2 to pierce through the tissue. Alternatively, the micro needle can be pressed against the sensory retina until it pierces through, at which point, vacuum can be applied to retain the retinal tissues away from the distal tip of the micro needle.

As the sensory retina is captured and held in place by the outer annular vacuum, a protected pocket 3c is created and the tissue is prevented from folding onto itself and occluding the micro needle tip, shown in FIG. 2. The distal opening of the piercing micro needle 2 now resides within this protected space, enabling the micro needle to aspirate the SRF without blockage by the sensory layer of retina. As the device is advanced forward towards the underlying retinal pigment epithelium (RPE) layer, the device will continue to aspirate SRF, represented by arrows 3d. Alternatively, the device can maintain its position as SRF is aspirated and the RPE will be pulled towards the sensory retina.

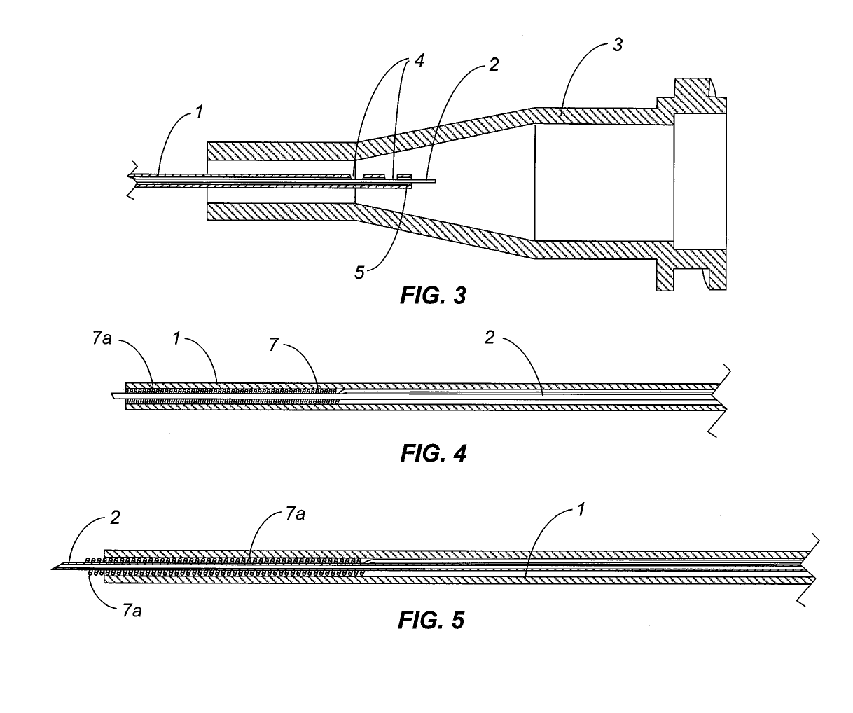

As shown in FIG. 3, the micro needle 2 runs the entire length of the main shaft 1 and beyond the proximal end of the main shaft 5. One or more holes or fenestrations 4 are formed near the proximal end of the main shaft. The micro needle is fixed in position by applying adhesive or a similar fixation method to the outer annular space between the proximal tip of the main shaft and the one or more fenestrations 4 drilled near the proximal end of the main shaft 5. The main shaft is inserted to the connection device 3 and fixed in position such that both the fenestrations 4 and the micro needle are in communication within the connection device. When vacuum or infusion is applied at the connection device 3, vacuum or infusion will be applied to both the outer annular space of the main shaft and within the micro needle.

In another embodiment, as shown in FIG. 4, a device is shown comprising a tissue blocking mechanism to prevent ingress of tissues into the outer annular space. The blocking mechanism may comprise of a coil, sheet apparatus with perforations or wire loop 7a within the outer annular space 7. The coil or loop may reside within the distal end of the outer annulus. When vacuum is applied to the device, the coil or loop blocks the entry of tissues into the annular space. By controlling the dimensions of the blocking mechanism within the annulus, the vacuum aspiration rate of the outer annulus can be reduced, thereby providing for a differential vacuum level between the outer annulus and the micro needle.

In another embodiment, as shown in FIG. 5, the blocking member 7a, such as a coil, may extend slightly beyond the distal end of the main shaft 1. When a vacuum is applied to the device, the tissues will apply pressure against the blocking member, causing the member to compress and retract, while simultaneously preventing injury to the tissues and blocking of the aspiration pathway.

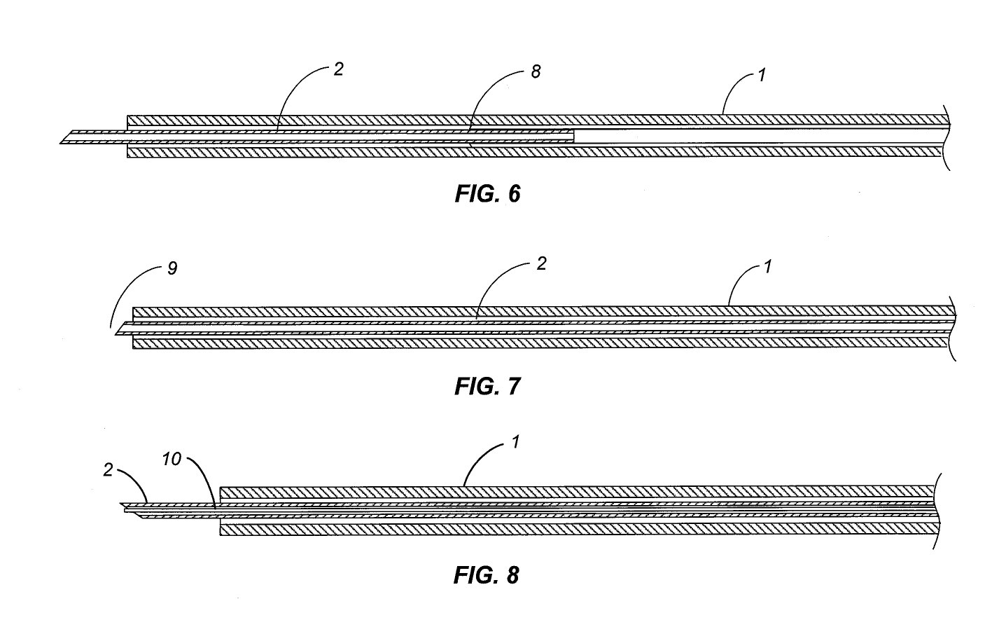

In another embodiment, as shown in FIG. 6, the device comprises an increased-diameter micro needle lumen 8 in the proximal non-tissue contacting portion of the main shaft 1. The increased diameter allows for maximization of the aspiration flow path.

In another embodiment, as shown in FIG. 7, the micro needle comprises a feature to facilitate entry into the subretinal space, such as a beveled distal tip 9 on the micro needle.

In another embodiment, as shown in FIG. 8, the device comprises a stiffening member 10, such as a small diameter metallic wire, disposed within the lumen of the micro needle 2 to help prevent kinking. Typically the micro needle may be fabricated of a polymer material, such as a polyimide, or a metal, such as, stainless steel. The wire may be fabricated from a high modulus material such as a metal, ceramic or structural polymer. The wire may be positioned within the lumen of the microneedle 2, or alternatively may be attached to the inner or outer wall of the main shaft 1.

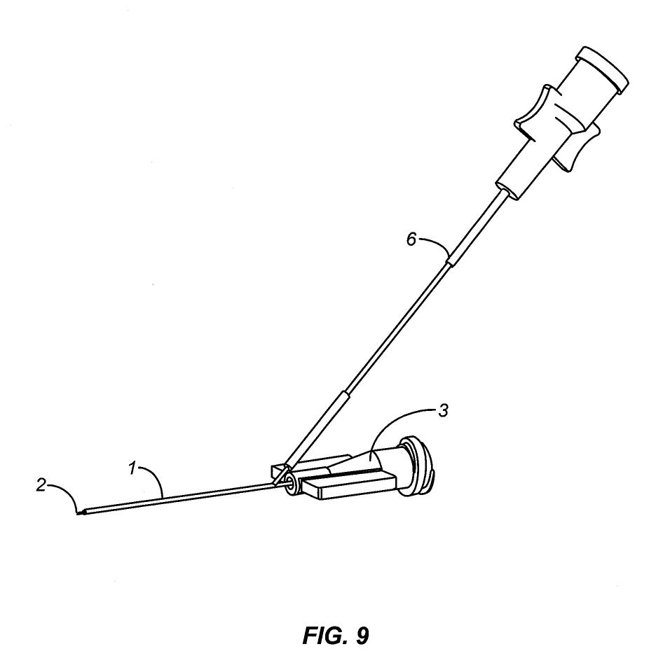

In another embodiment, as shown in FIG. 9, the device comprises a third hollow tubular member in communication with the annular space, an infusion line 6, disposed separately from the vacuum connection via device 3. The infusion line is designed to provide access to the outer annular space only, and is not in communication with the lumen of the micro needle 2. Following aspiration of the SRF, residual vacuum may keep the sensory retina attached to the outer annular space of the device. A slow, gentle infusion of a physiologically compatible medium, such as balanced salt solution, can be used to gently release the tissues from the tip of the device.

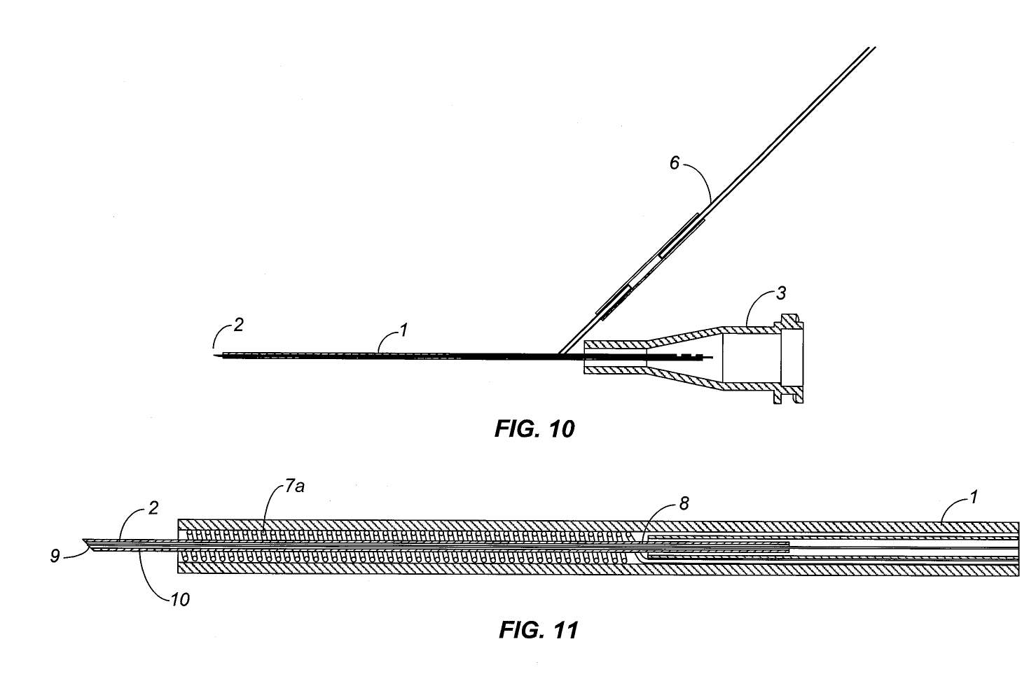

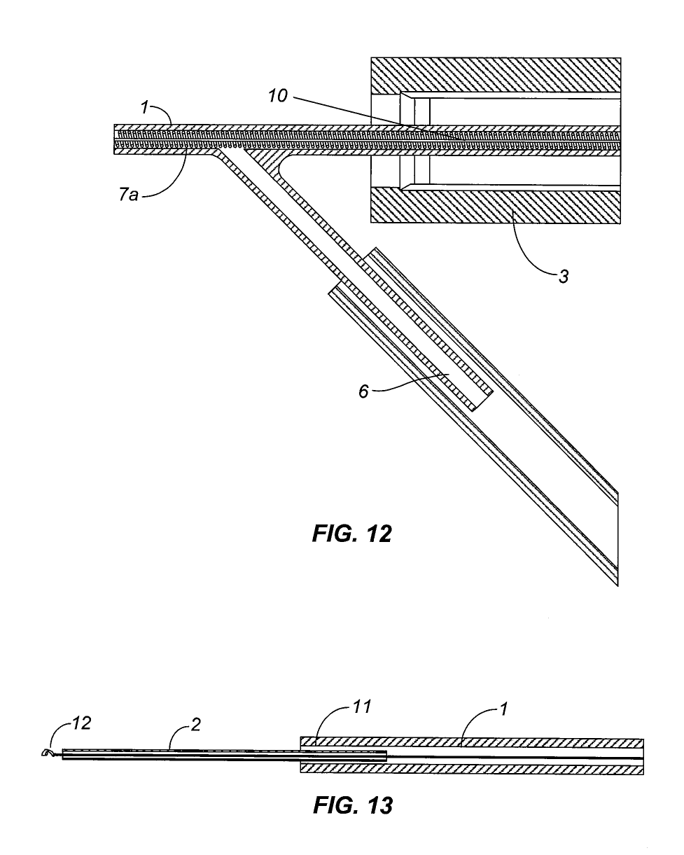

In several embodiments, as shown in FIG. 10, FIG. 11, and FIG. 12 the device comprises a combination of the aforementioned components. The device comprises a main shaft 1, a micro needle 2, a connection device 3 to attach the device to a vacuum source, an infusion line 6, a tissue blocking mechanism in the form of a coil 7a, an increased-diameter micro needle 8 to maximize aspiration, a beveled distal tip 9 on the micro needle to facilitate penetration into the tissues, and a stiffening member 10 to prevent kinking in the form of a wire.

In another embodiment, as shown in FIG. 13, the micro needle 2 protrudes from the main shaft 1 for a distance in the range of 0.01″ to 0.5″ and its proximal portion ends within the main shaft. A filler material 11, such as an adhesive, fills the void within the outer annular space. When the device is connected to a vacuum source, a vacuum is then applied to only the micro needle. A tissue guard 12 protruding from the micro needle will prevent occlusion of the micro needle by guarding the opening and preventing retinal tissue from collapsing into the micro needle. As shown in FIG. 13, the tissue guard may take on the form of a wire loop. Additional forms of the tissue guard may include variations, such as a coil disposed within the micro needle, a ball welded to the end of a wire, or a flat wire formed into a U at the distal tip.

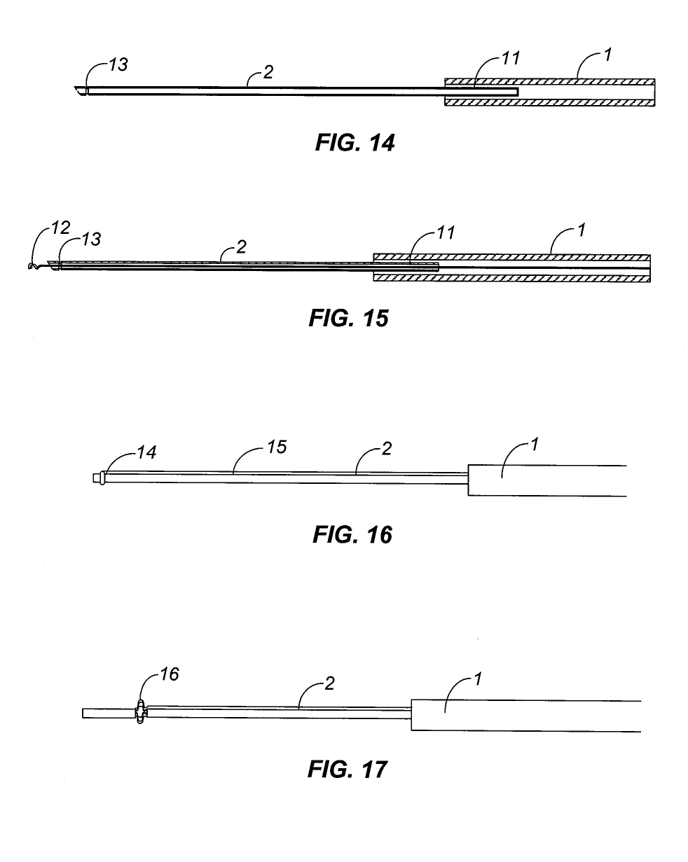

In another embodiment, as shown in FIG. 14, the micro needle 2 protrudes from the main shaft 1 for a distance typically of about 0.01″ to about 0.5″ and its proximal portion ends within the main shaft. A filler material 11, such as an adhesive, fills the void within the outer annular space. When the device is connected to a vacuum source, a vacuum is then applied to only the micro needle. One or more holes or fenestrations 13 are present near the distal opening of the micro needle. The holes range typically in size between about 0.0005″ to about 0.005″ and the center of the hole is typically a distance in the range of about 0.001″ to about 0.010″ from the distal edge of the micro needle. When vacuum is applied, the holes or fenestrations capture the retinal tissue and prevent ingress of the retinal tissue into the distal micro needle opening. The fenestrations may be formed in various patterns in order to control distribution of the retinal tissue.

In another embodiment, as shown in FIG. 15, the micro needle 2 typically protrudes from the main shaft 1 for a distance of about 0.01″ to about 0.5″ and its proximal portion ends within the main shaft. A filler material 11, such as an adhesive, fills the void within the outer annular space. When the device is connected to a vacuum source, a vacuum is then applied to only the micro needle. The micro needle has one or more holes or fenestrations 13 drilled near the distal opening of the micro needle. A tissue guard 12 having an atraumatic tip is disposed within the micro needle. Both the holes or fenestrations and tissue guard, in combination, will controllably capture and prevent the retinal tissue from occluding the distal opening.

In another embodiment, as shown in FIG. 16, the micro needle 2 typically protrudes from the main shaft 1 for a distance of about 0.01″ to about 0.5″ and its proximal portion ends within the main shaft. A balloon external tissue guard 14 is present near the distal opening of the micro needle. The distance between the distal opening of the micro needle and the edge of the tissue guard may typically range from 0.0″ to about 0.010″ in order to accommodate the range in sizes of retinal thicknesses and size of retinal detachments. The external tissue guard may have a collapsible design such that upon entry into subretinal space, the tissue guard does not injure the sensory retina. When the external tissue guard has penetrated the sensory retina, the external tissue guard may be deployed. The external tissue guard may be deployed using a user actuated mechanism internal or external to the micro needle. Furthermore, the external tissue guard may be deployed automatically when the device is present in the subretinal space. When the device is connected to a vacuum source, the external tissue guard will prevent the retinal tissue from occluding the distal opening by maintaining a guarded space near the micro needle opening. The external tissue guard may take on the form of a balloon, as shown in FIG. 16, which can be inflated when the external tissue guard is in the subretinal space. A lumen leading to the balloon but separate from the micro needle may be disposed within or external to the micro needle 15, such that infusion of a gas or fluid media would inflate the balloon without infusing media into the micro needle. Furthermore, aspiration of the media from the separate lumen would remove the media from the balloon and deflate the balloon.

In another embodiment, as shown in FIG. 17, the micro needle 2 protrudes from the main shaft 1 for a typical distance of about 0.01″ to about 0.5″ and its proximal portion ends within the main shaft. An external tissue guard 16 is present near the distal opening of the micro needle. The distance between the distal opening of the micro needle and the edge of the tissue guard may typically range from about 0.0005″ to about 0.010″. The external tissue guard may have a collapsible design. When the external tissue guard has penetrated the sensory retina, a sensing mechanism may allow for the external tissue guard to be automatically deployed. The sensing mechanism may be mechanical or electrical. A mechanical automatic deployment may occur when the distal opening of the micro needle contacts the RPE and choroid layer and the mechanical mechanism may take the form of slits 16 near the distal opening of the micro needle that allow the micro needle to flare out when contacting the RPE and choroid layer.

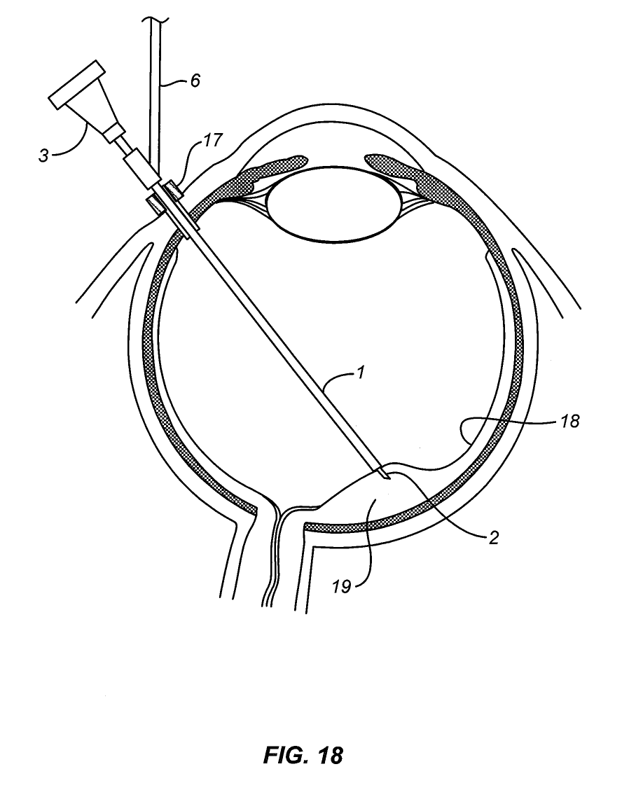

Referring to FIG. 18, a device is shown comprising an outer tubular member 1 as the first element, a smaller tubular member 2 following the same axis as the second element, and one or more connection devices 3 for introducing materials into the device or aspirating materials through the device and providing selective communication between the tubular members and other devices. A side arm 6 provides communication with various pathways created by the geometry of the tubular members. The device is inserted into the eye through a conventional sclerostomy port 17. While the sensory retina 18 is captured and held in place by the outer annular vacuum, a protected pocket 19 can be created beneath by gentle injection of balanced salt solution through the access shaft, creating a temporary retinal detachment that can be reversed at the end of the procedure if desired by aspiration of the injected fluid through the access shaft. The distal tip of the access shaft 2 shown residing within this protected space, enables direct access to the sensory layer of the retina, RPE and choroid.

The following examples are for illustration purposes and are not intended to limit the invention in any way.

### EXAMPLE 1

### Aspiration Device

A 25 gauge stainless steel hypotube (Small Parts, Inc) was used as the main shaft. Two holes were drilled at distances of 0.05″ and 0.12″ from the proximal edge of the hypotube. A third hole was drilled 1.15″ from the distal edge of the hypotube. A second 25 gauge stainless steel hypotube (Small Parts, Inc) was laser welded at an angle to provide a flow path to the third hole.

A polyimide tube with a lumen of 100 microns, an outer diameter of 125 microns, and a length of 0.25″ (Microlumen, Inc) was inserted for a distance of 0.05″ into another polyimide tube with a lumen of 165 microns, an outer diameter of 210 microns, and a length of 1.45″. Cyanoacrylate adhesive (Loctite 4011, Loctite, Inc) was applied to bond the two polyimide tubes together.

A nitinol coil with a length of 0.165″ and outer diameter of 250 microns was made on a coil winder using nitinol wire with a diameter of 0.0015″ (Fort Wayne Metals, Inc). The nitinol coil was placed over polyimide tube assembly, such that the additional nitinol wire extended towards the proximal portion. The polyimide tube assembly with the overlaid coil was then inserted into the main shaft and fixed with a cyanoacrylate adhesive, proximal to the two drilled holes. The distal tip of the polyimide tube assembly protruded from the main shaft, and the coil was captured within the main shaft such that the distal end of the coil was flush with the distal end of the main shaft.

A nitinol wire with a diameter of 0.0015″ was inserted into the polyimide tube assembly and fixed proximal to the main shaft by bonding the nitinol wire to the outer wall of a 22 gauge stainless steel hypotube with UV cure epoxy (Loctite 3341, Loctite, Inc). The 22 gauge stainless steel hypotube was welded over the proximal edge of the main shaft so as not to obstruct the two drilled holes, and a hole was drilled into the 22 gauge stainless steel hypotube through which the nitinol wire was threaded.

The main shaft was inserted into a luer fitting and fixed in position using UV cure epoxy. A Pebax tube was bonded to the infusion arm, and a luer fitting was bonded to the proximal end of the Pebax tube to provide fluid connection to the infusion arm.

### EXAMPLE 2

### Laboratory Testing with the Aspiration Device

A human cadaver eye was obtained from an eye bank. The cornea, the iris, the lens, and the vitreous were removed, providing access to the retina from the interior of the globe without significantly damaging the retina tissue, while also allowing for the retina to retain its original physiological attachments. Using existing post-mortem retinal detachments or creating a retinal detachment using phosphate-buffered saline injected through a needle inserted through the exterior of the globe into the subretinal space, experiments were conducted using the prototype.

The aspiration device from Example 1 was inserted into the subretinal space and a vacuum level in the range of 300 mm Hg to 600 mm Hg was applied. The retinal tissue was visibly captured by the outer annular vacuum, while fluid and tissue debris visibly migrated towards the micro needle. The device was capable of aspirating SRF until the re-apposition of the sensory retina and the underlying RPE and choroid occurred. The vacuum was turned off. Infusion of phosphate buffered saline into the infusion line helped release the device from the retina. A visual assessment of the access site after removal of the device only showed the entry site of the micro needle in the sensory retina. There was no apparent change to the tissue surround the entry site from the outer annular vacuum.

### EXAMPLE 3

### An Aspiration Device with External Tissue Guard

A 25 gauge stainless steel hypotube (Small Parts, Inc) was used as the main shaft and cut to a length of 1.25″. A 0.25″ length of polyimide tubing with an inner diameter of 0.0044″ and an outer diameter of 0.0056″ (Microlumen, Inc.) was used as the micro needle. Cyanoacrylate adhesive (Loctite 4011, Loctite, Inc.) was used to bond the micro needle within the main shaft, such that 0.20″ of the micro needle protruded from the main shaft. UV cure epoxy (Loctite 3341, Loctite, Inc.) was applied near the distal opening of the micro needle in the shape of a disc 360 degrees around the micro needle to act as a tissue guard. The disc had a diameter of 0.012″.

### EXAMPLE 4

### Laboratory Testing with an Aspiration Device with External Tissue Guard

A human cadaver eye was obtained from an eye bank. The cornea, the iris, the lens, and the vitreous were removed, providing access to the retina from the interior of the globe without significantly damaging the retina tissue, while also allowing for the retina to retain its original physiological attachments. Using existing post-mortem retinal detachments or creating a retinal detachment using phosphate-buffered saline injected through a needle inserted through the exterior of the globe into the subretinal space, experiments were conducted using the prototype.

The aspiration device with an external tissue guard from Example 3 was inserted into the subretinal space, such that the external tissue guard was in the subretinal space. A vacuum level in the range of 300 mm Hg to 600 mm Hg was applied. Aspiration of the SRF was visualized and the external tissue guard successfully prevented the occlusion of the polymer micro needle.

What is claimed is:

1. An apparatus for use with an eye, said apparatus comprising:
a first elongated tubular member having a proximal and a distal end and a lumen passing from said proximal end to said distal end;
a second elongated tubular member having a proximal end and a distal end having a pointed tip, disposed within said lumen of said first tubular member, said second elongated tubular member having a passage therethrough from said proximal end to said distal end;
an annular space within the lumen of said first elongated tubular member, annularly surrounding said second elongated tubular member wherein said passage and said annular space are in communication;
said distal end of said first elongated tubular member being open-ended and adapted to be placed in contact with a tissue surface whereby upon reduction of pressure within said annular space, said distal end of said first elongated tubular member seals to said tissue and said pointed tip penetrates said tissue and aspirates fluid material beneath said tissue into said passage from said distal end of said second elongated tubular member.

2. The apparatus according to claim 1 wherein said passage in said second elongated tubular member in communication with a device for aspirating fluids, suspensions, viscous solids or gases, through said passage.

3. The apparatus according to claim 1 wherein the distal end of said second elongated tubular member extends beyond the open distal end of said first elongated tubular member.

4. The apparatus according to claim 1 further comprising a blocking member disposed in said annular space at the distal end of said apparatus, said blocking member having a configuration sufficient to substantially prevent the ingress of tissues into said annular space through said open distal end without preventing fluid flow through said annular space.

5. The apparatus according to claim 4 wherein said blocking member comprises a coil.

6. The apparatus according to claim 4 wherein said blocking member comprises a loop.

7. The apparatus according to claim 4 wherein said blocking member comprises a perforated sheet.

8. The apparatus according to claim 7 wherein the perforations in said sheet have average diameters in the range from about 0.0001 inch to about 0.005 inch.

9. The apparatus according to claim 3 wherein said second elongated tubular member extends beyond the open distal end of said first elongated tubular member by about 0.0015 inch to about 0.125 inch.

10. The apparatus according to claim 1 wherein said second elongated tubular member comprises a polymer.

11. The apparatus according to claim 10 wherein said polymer comprises a polyimide.

12. The apparatus according to claim 1 wherein said first elongated tubular member comprises a metal.

13. The apparatus according to claim 12 wherein said metal comprises stainless steel.

14. The apparatus according to claim 1 further comprising a stiffening member disposed within said lumen.

15. The apparatus according to claim 14 where in said stiffening member comprises a wire.

16. The apparatus according to claim 1 further comprising a third hollow tubular member in communication with said annular space.

17. The apparatus according to claim 3 further comprising a tissue guard disposed within the passage of said second elongated tubular member that extends beyond the open distal end of said first elongated tubular member.

18. The apparatus according to claim 17 wherein said tissue guard comprises a wire loop.

19. The apparatus according to claim 17 wherein said tissue guard comprises a coil.

20. The apparatus according to claim 17 wherein said tissue guard comprises a wire having an atraumatic tip.

21. The apparatus according to claim 3 further comprising one or more fenestrations in said second elongated tubular member that extends beyond the open distal end of said first elongated tubular member.

22. The apparatus according to claim 21 wherein said fenestrations have a maximum diameter of in the range of about 0.0005 inch to 0.005 inch.

23. The apparatus according to claim 22 wherein the centers of said one or more fenestrations are a distance from the distal end of said second elongated tubular member in the range from about 0.001 inch 0.01 inch.

24. The apparatus according to claim 3 further comprising a tissue guard disposed external to said second elongated tubular member that extends beyond the open distal end of said first elongated tubular member.

25. The apparatus according to claim 24 wherein said tissue guard is collapsible.

26. The apparatus according to claim 24 wherein said tissue guard is disposed up to about 0.01 inches from the distal end of said second elongated tubular member that extends beyond the open distal end of said first elongated tubular member.

27. The apparatus according to claim 26 wherein said tissue guard comprises a balloon.

28. The apparatus according to claim 24 wherein said tissue guard comprises slits that expand when compressed.

29. The apparatus according to claim 24 wherein said tissue guard is expandable by activation.

30. The apparatus according to claim 29 further comprising a sensor for activating said tissue guard.

31. The apparatus according to claim 30 wherein said tissue guard is activated mechanically by said sensor.

32. The apparatus according to claim 30 wherein said tissue guard is activated electrically by said sensor.

33. The apparatus according to claim 30 wherein said tissue guard is adapted to activate to automatically expand upon penetration into the subretinal space.

34. The apparatus according to claim 1 wherein said first elongated tubular member is suitably sized to pass through a sclerostomy port.

35. The apparatus according to claim 1 wherein said distal end of said first elongated tubular member is adapted to contact a tissue surface in the interior of the eye.
